(19) United States
(12) Patent Application Publication
(10) Pub. No.: US 2019/0022005 A1
(43) Pub. Date: Jan. 24, 2019

(54) Multi-Drug Lipsomes to Treat Tumors

(71) Applicants: Henry J. Smith, Temecula, CA (US); James R. Smith, Laguna Niguel, CA (US)

(72) Inventors: Henry J. Smith, Temecula, CA (US); James R. Smith, Laguna Niguel, CA (US)

(21) Appl. No.: 16/115,973

(22) Filed: Aug. 29, 2018

Related U.S. Application Data
(62) Division of application No. 13/374,859, filed on Jan. 20, 2012.
(60) Provisional application No. 61/461,769, filed on Jan. 24, 2011.

(51) Int. Cl. A61K 9/127

(52) U.S. Cl. CPC A61K 9/127; A61K 9/1271

(57) ABSTRACT

A process for treating tumors by administering a mixture of cancer fighting drugs incorporated into a stabilized liposomal formulation. Each cancer drug is selected to target a different phase of the cell-cycle of the cancer cell thus expanding the number of cancer cells that can be killed at one time without compromising the safety of the patient. The stabilized multi-drug liposomes are designed to extravasate thru “leaky” blood capillaries supplying the tumor and enter the tumor tissue where they will accumulate over time and ultimately release the mixture of cancer drugs to kill surrounding tumor cells. The multi-drug liposomes are likewise unable to extravasate thru normal blood capillaries and will thus be less toxic to normal tissues.

### CROSS-REFERENCE TO RELATED APPLICATIONS

This divisional patent application claims priority to U.S. patent application Ser. No. 13/374,859 filed Jan. 20, 2012 and Provisional Patent Application 61/461,769 filed Jan. 24, 2011.

### STATEMENT RE: FEDERALLY SPONSORED RESEARCH/DEVELOPMENT

Not Applicable

### BACKGROUND

There are a wide variety of cancer drugs available to treat cancer. Cancer is characterized by uncontrolled cell division and the majority of small molecule cancer drugs are designed to affect cell-division or DNA synthesis and function. Although these drugs can kill cancer cells they are non-specific in their action and will also kill normal cells undergoing cell division. When patients are treated with a small molecule cancer drug by intravenous injection the drug quickly exits the bloodstream and distributes throughout the body causing undesirable side-effects such as nausea, vomiting, hair loss, anemia and susceptibility to infection.

Pharmaceutical companies screen thousands of compounds every year searching for new cancer drugs that will show superior efficacy and safety characteristics compared to current drugs. For the most part this has met with limited success with the newer drugs demonstrating only incremental improvement in safety or efficacy. The sequence of events in the development of a new cancer drug follows a fairly typical pattern; First thousands of compounds are screened for activity in vitro against a panel of different cancers. Those compounds showing activity against a particular tumor type are then screened for efficacy and safety against the same tumor type in animals. Those that pass are then tested in cancer patients with the same tumor type. The important point to note here is that current drug development programs focus primarily on the differentiated cell type of the tumor to be treated. For example, one particular drug will be developed, tested and registered with the FDA to treat a specific tumor type (e.g. breast cancer); while another drug will be developed and registered with the FDA to treat a different tumor type (e.g. prostate cancer).

In the invention described herein we teach an alternative approach to drug development that focuses on the “proliferative capacity” of the cancer cells making up the tumor rather than on the nature of its cellular differentiation and/or cell lineage. We believe that the “proliferative capacity” of the cancer cell (i.e. the speed of cell-division and the length of the different phases of the cell-cycle) are the prime factors to consider for the new class of cancer pharmaceuticals described in this invention. According to our theory the therapeutic efficacy of the novel class of pharmaceuticals described herein will be similar for tumors that have a similar “proliferative capacity” irrespective of the cell type from which they arose. For example, in terms of therapeutic response rapidly dividing breast cancer cells will have more in common with rapidly dividing lung cancer cells, and rapidly dividing prostate cancer cells, than with slowly dividing breast cancer cells. Therefore a pharmaceutical that is developed to treat rapidly dividing breast cancer cells will be expected to be also effective against rapidly dividing cancer cells of other tumor types such as rapidly growing lung cancer, and rapidly growing prostate cancer and rapidly growing colon cancer, and other rapidly growing tumors.

Another novel aspect of this invention is that instead of the conventional approach of screening for a new molecule cancer drug we consider the desirable features we would like to have in a cancer drug and we then construct a novel compound pharmaceutical composed of those desirable elements. Briefly, we teach the unique formulation of a novel class of multi-drug pharmaceuticals in which two or three or more different cancer drugs are combined into a stabilized liposomal formulation and administered to the cancer patient. Each drug is selected to target a different phase in the cell-cycle of the tumor cell. The administration of multiple different cancer drugs simultaneously into the patient will result in increased efficacy while at the same time their incorporation into liposomes will result in reduced cytotoxicity to the patient.

There are no prior teachings on the theory, design, and reduction to practice, of a novel class of multi-drug liposomal pharmaceuticals in which each of the component drugs is selected to target a different phase in the cell-cycle of the tumor cell.

### BRIEF SUMMARY

This invention describes the incorporation of multiple cancer drugs into a single stabilized liposome formulation in order to achieve improved efficacy and safety in treating tumors. The stabilized multi-drug liposomes will extravasate thru the “leaky” blood capillaries supplying the tumor and enter the tumor tissue where they will accumulate over time. Here the drugs are released to kill surrounding tumor cells. Typically, each of the component drugs selected will target a different phase of the cell-cycle of the cancer cell, thus expanding the number of cancer cells that can be killed at one time without compromising the safety of the patient.

### DETAILED DESCRIPTION

The prime characteristic of cancer cells is uncontrolled cell-multiplication. Cancer cells are either in the process of cell-division or they are preparing for cell-division. The tumor is composed of cancer cells that are in different phases of the cell-cycle. The cell-cycle can be broadly divided into four distinct phases: G1 phase (gap 1), S phase (DNA synthesis), G2 phase (gap2) and M phase (mitosis).

This invention teaches the theory, design, and reduction to practice, of a novel class of multi-drug liposomal pharmaceuticals that will focus on the “proliferative capacity” of the cancer rather than the cell type from which it arose.

In this invention “proliferative capacity” refers to the speed at which the cancer cell completes its cell division cycle and the duration of each phase of its cell-cycle (i.e. G1 phase, S phase, G2 phase and M phase).

The G1 phase is the first phase in the cell-cycle. During this phase the cell exhibits high biosynthesis of compounds required for cell growth and for DNA replication. Once the synthesis for DNA commences this marks the beginning of the S phase of the cell cycle. During the S phase the DNA of the cell is replicated so that by the end of the S phase all of the chromosomes have doubled with each former individual chromosome now represented by two sister chromatids. The S phase is followed by the G2 phase in which there is active biosynthesis of microtubules which play an essential role in the process of mitosis. During mitosis (M phase) the replicated chromosomes are segregated into two clusters and the cell completes its division into two daughter cells with each bearing its own replicated set of chromosomes.

There are a wide variety of pharmaceuticals to treat cancer. The majority of small molecule drugs affect cell-division or DNA synthesis and function. They can be classified as alkylating agents, antimetabolites, anthracyclines, plant akaloids, and topoisomerase inhibitors.

Alkylating agents are compounds that alkylate many nucleophilic functional groups. They impair cell function by forming covalent bonds with the amino, carboxyl, sulfhydryl, and phosphate groups in biologically important molecules. Cisplatin, carboplatin, and oxaliplatin, are alkylating agents. Other agents work by chemically modifying a cell's DNA. They include mechlorethamine, cyclophosphamide, chlorambucil, and ifosfamide.

Antimetabolites are chemical analogues to natural compounds utilized in cell metabolism. Purine analogues (e.g., fludarabine) inhibit the function of multiple DNA polymerases, DNA primase, and DNA ligase. Pyrimidine analogues (e.g., 5-fluorouracil) inhibits thymidylate synthase which is involved in DNA synthesis. Folic acid analogues (e.g., methotrexate) bind to and inhibits the enzyme dihydrofolate reductase (DHFR), and thus prevents the formation of tetrahydrofolate which is essential for purine and pyrimidine synthesis and thereby prevents DNA synthesis. One way or another these drugs inhibit DNA synthesis and thus target the S phase.

Anthracyclines are antibiotics that can also prevent cell division by disrupting the structure of DNA in two ways. They intercalate into the base pairs in the DNA minor grooves; and they can also cause free radical damage of the ribose in the DNA. The anthracyclines include daunorubicin, doxorubicin, epirubicin and idarubicin. These drugs target the S phase.

Plant alkaloids block cell division by preventing microtubule function and without their proper functioning cell division cannot occur. The main examples are vinca alkaloids (e.g., vincristine, vinblastine, vinorelbine) and taxanes (e.g., paclitaxel, docetaxel). Vinca alkaloids bind to specific sites on tubulin, inhibiting the assembly of tubulin into microtubules. The taxanes on the other hand enhance stability of microtubules, preventing the separation of chromosomes during anaphase. These drugs target the M phase.

Topoisomerase inhibitors are drugs that inhibit the enzymes that maintain the topology of DNA. Inhibition of type I or type II topoisomerases interferes with the transcription and replication of DNA. Type I topoisomerase inhibitors include camptothecins: irinotecan and topotecan. Type II inhibitors include amsacrine, etoposide, and teniposide. These drugs target the S phase.

The above examples of cancer drugs demonstrate that each drug targets a particular phase in the cell-cycle. It is important to note that the various phases of the cell cycle vary markedly in their duration. For most cells the G1 phase has the longest period and is the major fraction of the total cell cycle. It also shows the most variability from cell to cell and between different cell types. The S phase is fairly consistent in duration and has the next longest period. The M phase is also consistent in duration but only has a very short period compared to the overall cell cycle.

The duration of each phase is important. For example, the M phase is very short compared to the cell cycle and therefore the percentage of cancer cells in M phase will make up a small percentage of the total number of cancer cells. For example, if the cancer cells comprising the tumor have an average cell-cycle of 20 hours and an M phase of 30 minutes the percent of cells in M phase will be only 2.5 percent. Therefore a drug that targets the M phase will only affect a small percentage of cancer cells at one time. In order to increase the percentage of cancer cells killed it is required that an effective concentration of the drug remains within the tumor for an extended time so that as more and more cancer cells cycle into the M phase they will be exposed to the drug and be killed. Unfortunately most small molecule cancer drugs are either detoxified or eliminated from the body within a short period of time; and because they also distribute throughout all the body tissues only a small fraction actually reaches the tumor to be effective. In order to compensate for the limited bioavailability of these drugs they are therefore administered in large doses and/or on a repeated dosing schedule.

The same argument will apply to drugs that target the S phase. Although the S phase is longer than the M phase it is still a minor fraction of the duration of the total cell-cycle. Therefore drugs that target the S phase will only be cytotoxic to a fraction of the total number of cancer cells. As before, because of the limited bioavailability of these drugs they are administered in large doses and/or on a repeated schedule to compensate for this deficiency.

In order to increase the number of tumor cells killed there have been various attempts made to administer several cancer drugs concurrently using the argument that if one drug can kill a certain number of cells then two drugs given together will obviously kill a larger number of cells. Unfortunately, the increased cytotoxicity to the tumor cells is also accompanied by increased cytotoxicity to normal cells that negates this approach. This is why current chemotherapeutic programs typically comprise two or three or more cancer drugs that are given separately on different days according to a schedule that allows periodic intervals of rest for normal tissues to recover.

This invention teaches that contrary to conventional wisdom it is possible to administer two or more drugs simultaneously into the patient if the drugs are incorporated into a liposome or immunoliposome. Further this invention teaches that by selecting two or more drugs that target different phases of the cell-cycle to be incorporated into liposomes the resulting multi-drug liposomes will be safer and more effective than the predicate drugs administered in the conventional way. The following examples will serve to illustrate the principles underlying this invention and the advantages that will result.

This invention teaches the development of a novel class of pharmaceuticals whereby two or more cancer drugs are incorporated into a stabilized liposomal formulation with each drug selected to be cytotoxic to a different phase in the cell-cycle. Initially, the amount of each drug used will be in the same proportion relative to each other as they are currently prescribed. If one of the drugs is an insoluble drug it is incorporated into the lipid membrane of the liposome. Soluble drugs are encapsulated within the aqueous interior of the liposome. The mixture of lipids composing the liposomes are selected to have a transition temperature above 370 C so that the liposomes will not degrade prematurely when administered into the patient. In general liposomes that have a high transition temperature will be more resistant to degradation than liposomes having a lower transition temperature. High transition temperature liposomes will therefore release the drugs more slowly than those with lower transition temperatures. Polyethyleneglycol (PEG) polymer chains are also attached to the exterior surface of the liposomes which protects them from being recognized and destroyed by the liver or removed by the reticuloendothelial system. In some instances it may be advantageous to also attach a tumor targeting moiety (e.g., antitumor antibody or other binding agent) to the exterior surface of the liposomes in order to bind to tumor antigens within the tumor and/or facilitate internalization of the liposomes into the cancer cells. The diameter of the multi-drug liposomes is also critical to their selective localization within the tumor. The liposomes are manufactured to be of a certain standardized size in diameter between 100-400 nm, or preferably between 100-200 nm, or most preferably about 100 nm. Unlike conventional drugs, when the multi-drug liposomes are injected intravenously they will not be able to extravasate thru the endothelial pores of normal blood vessels and enter normal tissues. Neither will they be filtered out by the kidneys. Therefore they will be confined within the blood stream for an extended period of time. However, when the multi-drug liposomes reach the blood capillaries supplying the tumor they are able to extravasate thru the very enlarged endothelial pores of the tumor blood vessels and penetrate into the tumor tissue. Here they will degrade over time and release the drugs into the tumor thus exposing the tumor cells to a high concentration of the cancer drugs. As each drug targets a different phase in the cell cycle a higher percentage of the cancer cells will be killed. Further as the liposomes will release their contents over an extended period of time the cancer cells are continually exposed to these drugs for a long time. Therefore those cancer cells that escaped the initial cytotoxicity will continue on their cell cycle until they enter the specific phase where they are susceptible to the drug and are therefore killed. This will result in a significant increase in efficacy. Also as the multi-drug liposomes are unable to penetrate into normal tissues they will have greatly reduced harmful side-effects to the patient.

Liposomes

Liposomes are submicroscopic lipid vesicles. They can range in size from about 25 nm to over 1,000 nm in diameter. The unilamella liposomes of this invention are composed of a bilayer lipid membrane enclosing an aqueous center. The polar heads of the phospholipids are hydrophilic and therefore align and face the liquid exterior and also the liquid interior of the liposome. The hydrophobic regions (tails) of the phospholipid molecules line up within the lipid membrane. Soluble drugs can be enclosed within the aqueous center of the liposome while insoluble drugs are incorporated into the lipid bilayer of the liposome. Many cancer drugs are insoluble and must be dissolved in certain solvents before they can be administered. For example, paclitaxel has to be dissolved in castor oil and infused intravenously over a prolonged period to avoid triggering a toxic reaction in the patient. Incorporating the insoluble drug into liposomes obviates the necessity for a solvent and also allows the liposomal drug to be administered over a shorter period without triggering a toxic reaction.

Preparation of a Multi-Drug Liposome

The following examples illustrate the principle features underlying the variety of multi-drug liposomes that can be developed based on the different permutations of the cancer drugs that are incorporated into the liposome, and the different lipids used to prepare the liposome. Another variable component in the liposome formulation is if a targeting agent if attached to the exterior surface of the multi-drug liposome, and the nature of that agent. For example, the targeting agent is an antitumor antibody and the particular tumor antigen that is being targeted is a growth factor receptor or surface marker antigen on the cancer cell. These types of antibody coated liposomes are referred to as “immunoliposomes”. In addition there are other types of targeting agents such as binding peptides, or aptamers, or hormones, or cytokines, or growth factors, that can be substituted for and used in like manner to the antitumor antibodies.

The liposomes are prepared using a mixture of two or more of the following compounds: egg phosphatidylcholine (EPC), hydrogenated egg phosphatidylcholine (HEPC); soy phosphatidylcholine (SPC), hydrogenated soy phosphatidylcholine (HSPC), phosphatidylethanolamine (PE), phosphatidylglycerol (PG), phosphatidylinositol (PI), monosialoganglioside and sphingomyelin (SPM); distearoyl-phosphatidylcholine (DSPC), dimyristoylphosphatidylcholine (DMPC), dimyristoyl-phosphatidylglycerol (DMPG), dipalmitoylphosphatidylcholine (DPPC), and the derivatized vesicle forming lipids such as poly(ethyleneglycol)-derivatized distearoylphosphatidylethanolamine (PEG-DSPE) and poly(ethyleneglycol)-derivatized ceramides (PEG-CER). Typically, cholesterol in also included in the formulation.

This invention teaches the selection of two or more cancer drugs that target different phases in the cell-cycle of the cancer cell to be incorporated into the liposome. The cancer drugs are selected from the following categories of cancer drugs: these include the folate inhibitors, pyrimidine analogs, purine analogs, alkylating agents and antibiotics. Specific examples include acivicin, aclarubicin, acodazole, adriamycin, ametantrone, aminoglutethimide, anthramycin, asparaginase, azacitidine, azetepa, bisantrene, bleomycin, busulfan, cactinomycin, calusterone, caracemide, carboplatin, carmustine, carubicin, chlorambucil, cisplatin, cyclophosphamide, cytarabine, dacarbazine, dactinomycin, daunorubicin, dezaguanine, diaziquone, doxorubicin, epipropidine, etoposide, etoprine, floxuridine, fludarabine, fluorouracil, fluorocitabine, hydroxyurea, iproplatin, leuprolide acetate, lomustine, mechlorethamine, megestrol acetate, melengestrol acetate, mercaptopurine, methotrexate, metoprine, mitocromin, mitogillin, mitomycin, mitosper, mitoxantrone, mycophenolic acid, nocodazole, nogalamycin, oxisuran, peliomycin, pentamustine, porfiromycin, prednimustine, procarbazine hydrochloride, puromycin, pyrazofurin, riboprine, semustine, sparsomycin, spirogermanium, spiromustine, spiroplatin, streptozocin, talisomycin, tegafur, teniposide, teroxirone, thiamiprine, thioguanine, tiazofurin, triciribine phosphate, triethylenemelamine, trimetrexate, uracil mustard, uredepa, vinblastine, vincristine, vindesine, vinepidine, vinrosidine, vinzolidine, zinostatin and zorubicin. Also included are the toxins such as ricin and diptheria toxin

In one embodiment of this invention two or more soluble cancer drugs are encapsulated inside the liposome. Typically the exterior surface of the liposome is coated with a PEG polymer such as PEG-DSPE. One variation upon this embodiment is the attachment of a tumor targeting agent such as an antibody, or a binding peptide, or an aptamer, or a hormone, or a cytokine, or a growth factor to the surface of the liposome via a PEG link that will facilitate the selective localization of the liposomes within the tumor.

In one embodiment of this invention an insoluble cancer drug is incorporated into the lipid layer of the liposome and one or more soluble drugs are encapsulated inside the liposome. Typically the exterior surface of the liposome is coated with a PEG polymer such as PEG-DSPE. One variation upon this embodiment is the attachment of a tumor targeting agent such as an antibody, or a binding peptide, or an aptamer, or a hormone, or a cytokine, or a growth factor to the surface of the liposome via a PEG link that will facilitate the selective localization of the liposomes within the tumor.

It will be obvious to those of skill in the art that there are a wide variety of multi-drug liposomes that can formulated, and a wide variety of tumor targeting agents that can be employed, without departing from the spirit and scope of the teachings of this invention.

The following are examples of 1) the preparation of a multi-drug liposome and 2) the preparation of a multi-drug immunoliposome. For illustrative purposes we describe the incorporation of two cancer drugs where each targets a different phases in the cell-cycle of the tumor cell. The drugs selected are paclitaxel and danorubicin. Paclitaxel is an insoluble drug that targets the M phase of the cell-cycle, while danorubicin is a soluble drug that targets the S phase of the cell-cycle. Therefore the combination of these two drugs into a single liposomal formulation will be expected to result in superior efficacy compared to each drug given alone. Again, for illustrative purposes we describe the means whereby an insoluble drug and a soluble drug can both be combined into a single liposomal formulation to demonstrate the versatility of our invention and the wide variety of different cancer drugs that can be used.

### EXAMPLE 1

### Multi-Drug Liposome Incorporating an Insoluble Drug and a Soluble Drug

The liposome is composed of a mixture of soy phosphatidylcholine (SPC), hydrogenated soy phosphatidylcholine (HSPC), cholesterol and poly(ethyleneglycol)-derivatized distearoylphosphatidylethanolamine (PEG5000-DSPE) in the following molar ratios: 10/10/2/0.5. The lipid components are mixed together in a round bottomed flask and dissolved in a chloroform/alcohol solution. Typically there is approx. 25 mg lipid/ml organic solvent. For lipid soluble drugs such as paclitaxel the molar to lipid ratio is typically less than 5. The drug is dissolved in a small volume of chloroform/alcohol solution and added to the lipid mixture. The flask is then attached to a rotary vacuum evaporator and the drug/lipid solution is thoroughly dried under vacuum until a lipid film is formed on the interior surface of the flask. The dried lipid film is then hydrated with a daunorubicin solution maintained at 600 C and vigorously sonicated which causes the formation of liposomes encapsulating some of the daunorubicin solution within the interior of the liposome. The drug liposomes are then repeatedly extruded using a commercial homogenizer/extruder using graduated membranes of decreasing pore size from 500 nm to 100 nm. This results in unilamella liposomes having a controlled diameter of about 100 nm. The process is maintained at 600 C throughout. The drug liposomes are then cooled to room temperature and the unencapsulated daunorubicin is separated from the multi-drug liposomes using column chromatography or dialysis. The multi-drug liposomes are stored at 40 C or lyophilized and kept at −200 C for longer term storage. Lyophilized liposomes are reconstituted to original volume using distilled water or physiological solution suitable for injection or infusion before use.

An alternative method of encapsulating soluble drugs is to load the drug into preformed liposomes using a pH gradient method or an ammonium sulphate gradient method. Briefly, for the pH gradient method the lipid soluble drug is co-dissolved with the lipid mixture as before and dried under vacuum to form a lipid film. The lipid film is rehydrated using an acidic buffer such as citric acid maintained at 600 C and sonicated to prepare liposomes encapsulating the acidic buffer within the interior of the liposome. The liposomes are then extruded thru a commercial homogenizer/extruder using graduated membranes of decreasing pore size from 500 nm to 100 nm. This results in unilamella liposomes having a controlled diameter of about 100 nm. The process is maintained at 600 C throughout. The drug liposomes are then cooled to room temperature and the unencapsulated acidic buffer is separated from the liposomes using column chromatography or dialysis. The liposomes are then suspended in a solution of daunorubicin that has a higher pH than the interior of the liposome. This causes the amphiphilic drug outside the liposome to migrate and concentrate within the interior of the liposome. The process is maintained at 600 C to facilitate the migration of the drug across the lipid membrane of the liposome. The multi-drug liposomes are then cooled to room temperature and the unencapsulated daunorubicin is separated from the multi-drug liposomes using column chromatography or dialysis.

Another method of loading the soluble drug into the preformed liposomes is the ammonium sulphate gradient method. Briefly, the lipid soluble drug is co-dissolved with the lipid mixture and dried under vacuum to form a lipid film as described earlier. The lipid film is rehydrated using a solution of ammonium sulphate maintained at 600 C and sonicated to prepare liposomes encapsulating the ammonium sulphate within the interior of the liposome. The liposomes are then extruded thru a commercial homogenizer/extruder using graduated membranes of decreasing pore size from 500 nm to 100 nm. This results in unilamella liposomes having a controlled diameter of about 100 nm. The process is maintained at 600 C throughout. The drug liposomes are then cooled to room temperature and the unencapsulated ammonium sulphate is separated from the liposomes using column chromatography or dialysis. The liposomes are then suspended in a solution of daunorubicin. The ammonium ion within the interior of the liposome will migrate out of the interior causing the amphiphilic drug outside the liposome to migrate and concentrate within the interior of the liposome. The process is maintained at 600 C to facilitate the migration of the drug across the lipid membrane of the liposome. The multi-drug liposomes are then cooled to room temperature and the unencapsulated daunorubicin is separated from the multi-drug liposomes using column chromatography or dialysis.

The methods of preparing liposomes and of encapsulating soluble drugs within the liposome is well-known to those of skill in the art and are included within the scope of this invention. Similarly the methods of preparing liposomes and incorporating insoluble drugs within the lipid layer of the liposome are also well-known to those of skill in the art and are included within the scope of this invention.

### EXAMPLE 2

### Multi-Drug Immunoliposome Incorporating an Insoluble Drug and a Soluble Drug

The multi-drug immunoliposomes are prepared in the same manner as the multi-drug liposomes as described earlier with the following differences. In addition to the lipid mixture as described before a derivatized PEG conjugated lipid bearing a maleimide site (MAL) at the distal end of the PEG is included. For example the lipid mixture used to prepare the immunoliposome will consist of the following components: soy phosphatidylcholine (SPC), hydrogenated soy phosphatidylcholine (HSPC), cholesterol, and poly(ethyleneglycol)-derivatized distearoylphosphatidylethanolamine (PEG5000-DSPE), and poly(ethyleneglycol)-derivatized distearoylphosphatidylethanolamine with a malemide active site on the PEG (MAL-PEG2000-DSPE) in the following molar ratios: 10/10/2/0.5/0.025.

To prepare a tumor targeting multi-drug immunoliposome the Fab fragment prepared from a purified tumor targeting antibody is attached to the maleimide active site on the MAL-PEG2000-DSPE moiety anchored to the exterior surface of the multi-drug liposome. The tumor targeting antibody may be a polyclonal antibody, or a monoclonal antibody, or a recombinant protein. To avoid a patient reaction to the administered antibody a “humanized” monoclonal antibody or a fully human “phage display” recombinant antibody is preferred. The antitumor antibody is purified using standard laboratory methods such as salt-fractionation and/or protein A binding followed by immunoaffinity chromatography in which the antibody is allowed to bind to the target antigen immobilized on a matrix such as agarose beads followed by elution with an elution buffer such as glycine-HCl pH 2.5. The eluted antibody is brought to neutral pH and treated with the enzyme papain that cleaves the antibody molecule into two Fab fragments and one Fc fragment. The Fc fragment is removed by binding to Protein A and the remaining purified Fab fragment is conjugated to the maleimide site of a PEG-DSPE molecule that is anchored to the exterior surface of the liposome. The Fab fragment becomes attached in such a manner that its antigen binding site is not blocked but is free to bind to its respective antigen present within the tumor. The methods of antibody purification and Fab preparation are well known to those of skill in the art and are included within the scope of this invention.

An alternative method of attaching the Fab to the liposome is the “post-insertion method”. In this method the multi-drug liposomes are prepared as described earlier but with the MAL-PEG-DSPE omitted from the lipid mixture. The Fab is prepared as described earlier and then allowed to bind to the MAL-PEG-DSPE in a separate reaction. The Fab-MAL-PEG-ESPE moiety is then allowed to react with the formed multi-drug liposomes at a temperature that is higher than the transition temperature of the mixed lipids whereupon the DSPE end of the moiety will insert into the lipid layer of the liposomes thus anchoring the Fab to the liposome thru the PEG link. The Fab fragment becomes attached in such a manner that its antigen binding site is not blocked but is free to bind to its respective antigen present within the tumor.

In one embodiment of this invention the tumor targeting antibody is an anti-HER 2 antibody that will target Human Epidermal Growth Factor 2 receptors (HER2) that are over-expressed in some breast cancers. HerceptinR (trastuzumab) is a commercially available humanized monoclonal antibody that targets HER2 and there are biosimilar versions being developed. Anti-HER2 antibody and biosimilar versions can be used to prepare multi-drug immunoliposomes using the general methods described in this invention. Multidrug immunoliposomes prepared using anti-HER2 antibody will have the capacity to bind to breast cancer cells and anchor the immunoliposomes within the tumor. They will also have the additional advantage that by binding to the cancer cells they can either kill or inhibit its growth. It is also postulated that this effect is enhanced due to the bound immunoliposomes being internalized by the cancer cell thus delivering the drug directly into the cancer cell. Multi-drug immunoliposomes prepared using anti-EGFR2 antibody may therefore be the preferred pharmaceutical in treating breast cancer.

In one embodiment of this invention the tumor targeting antibody is an antibody that will target Human Epidermal Growth Factor 1 receptors (EGFR). ErbituxR (cetuximab) is a commercially available chimeric human/mouse monoclonal antibody that will target EGFR over-expressed in colorectal cancer and squamous cell carcinoma of the head and neck. VectibixR (panitumumab) is a fully human monoclonal antibody that also targets EGFR in metastatic colorectal cancer, and there are also biosimilar versions beings developed. Anti-EGFR antibody and biosimilar versions can be used to prepare multi-drug immunoliposomes using the general methods described in this invention. Multi-drug immunoliposomes prepared using anti-EGFR antibody will have the capacity to bind to the cancer cells and anchor the immunoliposomes within the tumor. They will also have the additional advantage that by binding to the cancer cells they can either kill or inhibit its growth. It is also postulated that this effect is enhanced due to the bound immunoliposomes being internalized by the cancer cell thus delivering the drug directly into the cancer cell. Multi-drug immunoliposomes prepared using anti-EGFR antibody may therefore be the preferred pharmaceutical in treating colorectal cancer and squamous cell carcinoma of the head and neck.

In one embodiment of this invention the tumor targeting antibody is an autoimmune antinuclear antibody (ANA) that targets the extracellular nuclear material that is present in the necrotic regions of solid tumors. The ANA is collected from patients with systemic lupus erythematosus (SLE) and purified using salt-fractionation and immunoaffinity methods. The Fab fragment of the antibody is prepared and attached to the multi-drug liposome thru a MAL-PEG-DSPE link as described earlier. When multi-drug immuoliposomes prepared in this manner are injected into cancer patients the ANA immunoliposomes will concentrate within the areas of necrosis where the drugs are released over time. As almost all solid tumors will have areas of necrosis the ANA immunoliposomes may be utilized to treat a wide variety of different types of solid tumors.

There are a growing number of new antitumor antibodies being developed that can be used to prepare multi-drug immunoliposomes. For example, many antitumor antibodies are known to target certain cell surface markers such as the CD antigens present on tumor cells. These can also be attached to multi-drug liposomes using the general principles outlined in this invention.

In this invention the term antitumor antibody refers to the whole antibody molecule, and/or the binding fragments Fab and F(ab′)2; and/or to recombinant binding proteins such as scFv.

It will be obvious to those of skill in the art that there are other known compositions of immunoliposomes that can be prepared and a variety of methods for manufacturing them. Likewise there are a variety of other known tumor targeting agents such as aptamers, binding peptides, hormones, growth factors and cytokines known to those of skill in the art that may be employed in like manner without departing from the spirit and scope of this invention.

Discussion

This invention teaches that cancer therapy using the novel pharmaceuticals described herein will not be based on the tumor type but will instead focus on the proliferative capacity of the tumor. This will include the cell-cycle time of the cancer cells, and it is our thesis that irrespective of the cell type from which they arose those cancers exhibiting similar proliferative capacities will be susceptible to the same multi-drug liposomal formulation. We predict for example, that rapidly dividing breast cancer cells and rapidly dividing lung cancer cells will both show a similar response to a particular multi-drug liposomal formulation; and this will be different from the response shown by slow dividing breast cancer cells and slow dividing lung cancer cells. We believe that eventually there will be developed one set of multi-drug liposomal formulations for cancers with rapid cell division, and another set of multi-drug liposomal formulations for cancers with slow cell division. The selection of particular drugs to be incorporated into the multi-drug liposomal formulation will also be guided by the duration of the various phases of the cell cycle of the cancer cells. For example, rapidly dividing cancer cells will be more susceptible to drugs that target the M phase and the S phase while slow dividing cancer cells may require the addition of a drug that targets the G1 phase. The tumor targeting liposomes will be further influenced by their targeting capacity to deliver the drugs to certain tumor types. Other factors to be considered will be the residence time that the drug is present and bioavailable within the tumor.

This invention teaches the development of a novel class of pharmaceuticals where each novel pharmaceutical is composed of a mixture of two or more cancer drugs incorporated into a liposomal formulation; with each drug targeting a different stage in the cell-cycle. Soluble cancer drugs are enclosed within the aqueous interior of the liposome while insoluble drugs are incorporated into the lipid bilayer membrane of the liposome.

This invention teaches that the multi-drug liposomal pharmaceuticals described herein will behave very differently from the predicate drugs with regard to their bioavailability and biodistribution. For example, under the current cancer therapy programs when small molecule drugs are administered intravenously they may be rapidly detoxified by the liver and/or filtered out thru the kidneys. They will also rapidly exit the blood system and distribute throughout the body tissues where they will be cytotoxic to dividing cells within the tumor and also to dividing normal cells within normal tissues. Therefore the dosage of a single cancer drug that can be given is limited by the maximum cytotoxicity tolerated by the patient; and it is for this reason that conventional treatment protocols recommend using various chemotherapeutic regimens in which different cancer drugs are administered according to a specified schedule.

In this invention we teach that the pharmacological attributes of conventional small molecule drugs no longer apply when they are combined and incorporated into liposomes. First, by incorporating the drugs within a stabilized liposomal formulation the drugs are protected from detoxification by the liver or removal by the reticuloendothelial system. Second, the multi-drug liposomes are manufactured to be between 100-200 nm in diameter. This makes them too large to be filtered out by the kidney or to extravasate thru normal blood vessels and therefore they will remain in the blood circulation for an extended period of time. They are however, still small enough to pass thru the endothelial pores of “leaky” blood vessels supplying the tumor and enter the tumor tissue where they will accumulate over time. Here the liposomes will break down releasing the drugs into the surrounding tumor tissue where they will have the most cytotoxic effect. In this context it should be noted that the release of the various component drugs incorporated into the liposome will be greatly influenced by whether they were encapsulated within the liposome or incorporated into the lipid layer of the liposome. For example, encapsulated drugs will be released rapidly as soon as the liposome begins to leak; while drugs incorporated into the lipid layer will take a longer time to be released from the lipid matrix. The selection of a soluble drug combined with an insoluble drug in the liposome will therefore result in an increased time of exposure of the tumor tissue to the cytotoxic effects of the combined drugs. At the same time as the multi-drug liposomes are too large to pass thru normal blood capillaries and enter normal tissues their cytotoxicity to normal tissues will be greatly reduced. The end-result is that multi-drug liposomes will have a superior efficacy and safety profile compared to conventional drugs.

In several embodiments of this invention we describe the rationale and method of attaching a tumor targeting agent to the multi-drug liposomes; and why they may be preferred in treating those tumors expressing the corresponding antigen.

Each different mixture of drugs incorporated into a liposomal and/or immunoliposomal formulation represents a novel pharmaceutical. Depending upon the type of tumor and medical history of the patient one or more of these multi-drug liposomal formulations can be administered according to a specified dosage and schedule that is designed to maximize cytotoxicity to the tumor without compromising safety.

This invention also teaches that because of the complexity of using multiple drugs to treat the tumor; and because each tumor is as individual as the person from whom it arose it is impossible to predict the therapeutic contribution of each drug to the overall efficacy and safety of the multi-drug liposomes. Therefore until such time as we are able make these predictions accurately we propose that the selection and dosages of the drugs to be incorporated in the multi-drug liposome will be done empirically based on the known pharmacological profile of the individual drugs given separately. Further, it is also well-known that incorporation of drugs into liposomes will alter their bioavailability and biodistribution profile so that in general a higher amount of the liposomal drug reaches the tumor compared to the free drug. Therefore we also propose that initially the dosage of each drug incorporated into the multi-drug liposome formulation be limited to a safe fraction of its known therapeutic dose. These dosages can then be increased or decreased according to additional studies and clinical experience.

This invention teaches an alternative means of cancer treatment utilizing multi-drug liposomes targeting different phases of the cell-cycle of the cancer cell. It explains the rationale and describes the process for developing this class of novel pharmaceuticals. To those accustomed to conventional pharmacology in which each novel drug is extensively and exhaustively studied before it is used, the means by which the novel pharmaceuticals described herein were designed and developed are not done according to the rules. We would argue that in reality it is impractical, if not impossible, to develop a multi-drug pharmaceutical according to conventional pharmacological principles because of the complex interaction of the various drugs utilized when they are formulated into a single compound pharmaceutical and selectively delivered to the tumor site. Nevertheless we believe that it is possible to formulate a multi-drug pharmaceutical based on prior knowledge of the characteristics of each of its constituents and to make a reasonable estimate regarding its potential efficacy and safety. We acknowledge that it will take additional research and clinical studies to validate this approach.

At the same time it is important to keep in mind that while orthodox pharmacological research will continue to search for the perfect drug that will only kill cancer cells without harming normal cells, there are thousands of patients who are suffering and dying from cancer that may benefit from the novel pharmaceuticals derived from the teachings of this invention. In this context we should heed the advice of a noted philosopher1 and “not let our desire for the perfect be the enemy of the good”.

This invention teaches the novel approach of using multi-drug liposomes and multi-drug immunoliposomes to treat tumors. It also teaches the use of other tumor targeting agents such as aptamers, binding peptides, hormones, cytokines and growth factors that can be similarly employed to deliver the tumor targeting multi-drug liposomes to the tumor. The examples provided herein are for illustration and discussion and not for limitation. Of course, variations on these described examples and embodiments will become apparent to those of ordinary skill in the art upon reading the foregoing description. We expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention. It is to be understood that the embodiments of the invention disclosed herein are illustrative of the principles of the present invention. Thus, by way of example, but not of limitation, alternative configurations and revisions of the present invention may be utilized in accordance with the teachings herein and are considered to be within the spirit and scope of this invention.

1-13. (canceled)

14. A method for forming a pharmaceutical composition for targeting tumor cells based upon their proliferative capacity, the method comprising the steps of:
selecting a first cytotoxic drug based upon that first cytotoxic drug selectively targeting a first phase in the cell-cycle of tumor cells;
selecting a second cytotoxic drug based upon that second cytotoxic drug selectively targeting a second phase in the cell-cycle of tumor cells, the second phase in the cell-cycle of tumor cells being different from the first phase in the cell-cycle of tumor cells; and
enclosing the first and second cytotoxic drugs within a liposome;
wherein the first cytotoxic drug is water soluble and enclosed within an aqueous interior of the liposome; and
wherein the second cytotoxic drug is lipid soluble and incorporated into a lipid bilayer of the liposome.

15. The method of claim 14, wherein the liposome further comprises a tumor targeting agent attached to the exterior surface of the liposome.

16. The method of claim 15, wherein the tumor targeting agent is selected from the group consisting of an antibody, a binding peptide, an aptamer, a hormone, a cytokine, a growth factor, and a compound capable of binding to the surface of the tumor cell.

17. The method of claim 14, wherein the first cytotoxic drug and the second cytotoxic drug are small molecule drugs that affect cell-division and/or DNA synthesis and function.

18. The method of claim 17 wherein at least one of the first cytotoxic drug and the second cytotoxic drug are selected from the group consisting of alkylating agents, antimetabolites, anthracyclines, plant alkaloids and topoisomerase inhibitors.

19. The method of claim 14 wherein the liposome is comprised of a mixture of one or more compounds selected from the group consisting of: egg phosphatidylcholine (EPC), hydrogenated egg phosphatidylcholine (HEPC); soy phosphatidylcholine (SPC), hydrogenated soy phosphatidylcholine (HSPC), phosphatidylethanolamine (PE), phosphatidylglycerol (PG), phosphatidylinositol (PI), monosialoganglioside and sphingomyelin (SPM); distearoyl-phosphatidylcholine (DSPC), dimyristoyl-phosphatidylcholine (DMPC), dimyristoyl-phosphatidylglycerol (DMPG), and dipalmitoylphosphatidylcholine (DPPC), poly(ethyleneglycol)-derivatized distearoylphosphatidylethanolamine (PEG-DSPE), poly(ethyleneglycol)-derivatized ceramides (PEG-CER), and cholesterol.

20. The method of claim 19 wherein the liposome is stabilized by attaching on or more PEGn-DSPE polymer chains to the exterior surface of the liposome, wherein “n” is the molecular weight of the polymer chains and exceeds 2,000 daltons.

21. The method of claim 16 wherein the targeting agent is an anti-epidermal growth factor 1 receptor antibody, or an aptamer or binding peptide that targets epidermal growth factor 1 receptor.

22. The method of claim 16 wherein the selected targeting agent is an anti-human epidermal growth factor 2 receptor antibody, or an aptamer or binding peptide that targets human epidermal growth factor 2 receptor.

23. The method of claim 16 wherein the selected targeting agent is an anti-nuclear antibody, an aptamer, or a binding peptide, and targets one or more nuclear materials released from dead cells within a tumor selected from the group consisting of: dsDNA, ssDNA, ENA/RNP, Sm and DNP.
